(19) United States
(12) Patent Application Publication
(10) Pub. No.: US 2021/0137542 A1
(43) Pub. Date: May 13, 2021

(54) MEDICAL DEVICE FOR RETRIEVAL OF CLOTS USING AN ELECTRICALLY CHARGED STENT STRUCTURE

(71) Applicants: Thomas James OXLEY, New York, NY (US); Nicholas Lachlan OPIE, Parkville (AU)

(72) Inventors: Thomas James OXLEY, New York, NY (US); Nicholas Lachlan OPIE, Parkville (AU)

(21) Appl. No.: 17/093,196

(22) Filed: Nov. 9, 2020

Related U.S. Application Data
(60) Provisional application No. 62/932,935, filed on Nov. 8, 2019.

(51) Int. Cl. A61B 17/221; A61B 34/00

(52) U.S. Cl. CPC A61B 17/221; A61M 2025/0042; A61B 2034/731; A61B 34/73

(57) ABSTRACT

Devices, methods and systems for using an electrically conductive stent structure to restore blood flow in an occluded vessel.

### RELATED APPLICATIONS

This application claims the benefit of U.S. Provisional Application No. 62/932,935 filed on Nov. 8, 2019, the entire contents of which are incorporated by reference.

### TECHNICAL FIELD OF THE INVENTION

The present invention relates to medical devices for removal of an obstruction, such as a clot or thrombus from vessel, typically a vessel that supplies blood to regions of the brain or a clot located in any other vessel, such as a pulmonary emboli.

### BACKGROUND OF THE INVENTION

The use of stent type structures to restore blood flow within the cerebral vasculature, where a clot, thrombus, or other obstruction blocks blood flow causing an ischemic stroke are increasing in frequency. When an individual suffers from an ischemic stroke, regions of the brain do not receive oxygenated blood from the heart and lungs. The lack of blood flow also prevents the removal of carbon dioxide and cellular waste from the affected brain tissue. Blockages that interfere with the supply of blood eventually result in permanent brain damage since the disruption of blood flow can cause irreversible brain cell death. Therefore, a patient suffering from an ischemic stroke must have the obstruction removed as soon as possible in order for recovery from the stroke.

The use of stent retrievers to remove the obstruction can increase positive outcomes for patients suffering an ischemic stroke. Typically, a physician advances the stent or retrieval structure into the cerebral region of the vasculature. When the retrieval device is located in the vessel of interest, the physician deploys a device for retrieval of the obstruction causing the blockage. Typically, the physician deploys the device directly into the obstruction, but the device can be deployed proximal or distal to the obstruction.

The removal of obstruction presents its own risks since the procedure can produce debris caused by disturbance or breaking of obstruction. The debris can often migrate to smaller vessels where navigation of a retrieval device is difficult if not impossible. In some cases, it is difficult or impossible to know if any debris migrates from the obstruction.

Use of a retrieval device often requires the physician to expand or position the clot retrieval device within the obstruction and then navigate the device and enmeshed obstruction to a guide catheter for removal from the body. Handling of the obstruction in this manner can also cause debris to break from the obstruction and cause blood flow blockage in smaller vessels.

In view of the above, there remains a need for an improved device that reduces the risk of debris from migrating during a surgical procedure intended to restore blood flow within a patient experiencing ischemic stroke.

Recently, stents and stent type devices have been discussed for use in restoring control to individuals that suffer from various neuromuscular disorders where control of limbs is severely impaired. In many of these patients, however, the portion of the brain responsible for movement remains intact, and it is disease and trauma to the spinal cord, nerves and muscles that limit mobility, function and independence. For these people, the ability to restore lost control at even a rudimentary level could lead to a greatly improved quality of life.

For example, commonly assigned U.S. patent application Ser. Nos. 15/957,574 and 16/164,482 disclose stent based devices that can record and stimulate cortical tissue when placed in the vasculature of the brain. Such devices use blood vessels as a conduit to the brain and provide for improved intravascular electrodes, telemetry circuitry and implantation positions that are capable of more efficiently transmitting and receiving electrical energy between vessels and external circuitry, while minimizing the occlusion of blood flow. While such devices are demonstrating promise in improving BCI for control of external devices. The stent-based devices can offer therapeutic advantages to meet a number of surgical needs.

It is known that all biological cells and surfaces of the human body carry an electrical charge. The magnitude of this charge is determined not only by the characteristics of the cells and particles themselves, but also by the liquid or solid in which they are immersed. Particles within blood carry a negative charged. It is also known that an incision into a vessel will result in a positive voltage at the incision site. Experiments have shown that if the incision site remains negatively using an electrical current, coagulation of blood takes longer at the site. If the incision site is provided with a positive voltage, clotting at the site accelerates. In a laboratory setting two oppositely charged electrodes that are immersed in blood form a clot at the positive electrode only. If the procedure is carried out correctly, the blood surrounding the negative electrode will have highly effective anticoagulant properties.

Therefore, there remains a need to control an electrical charge at the site of an occlusion within the brain can assist in controlling debris and/or thrombosis during the removal of the stent as well as produce areas within the vessel that provide anticoagulant properties.

### SUMMARY OF THE INVENTION

According to the present invention, there is provided a medical device for use in a clot/obstruction removal procedure intended to treat ischemic stroke, where the device can produce various electrical effects at and/or near the site of the occlusion.

In one variation, the present invention includes methods for restoring blood flow in a blood vessel occluded by an obstruction at an occlusion site in the blood vessel. For example, such a method can include advancing a microcatheter into the blood vessel, the microcatheter containing an expandable stent structure; deploying the expandable stent structure within the blood vessel such that a portion of the expandable stent structure embeds into the obstruction; applying energy to the expandable stent structure to cause a first electrical effect along a first portion of the expandable stent structure and a second electrical effect along a second portion of the expandable stent structure, within the blood vessel, where the first electrical effect varies from the second electrical effect and where at least the first electrical effect or the second electrical alters an attraction of the obstruction to the expandable stent structure; manipulating the expandable stent structure to dislodge at least a portion of the obstruction from the occlusion site; and withdrawing the expandable stent structure and at least the portion of the obstruction from the vessel allowing blood flow to resume within the blood vessel.

In one variation of the method, where the first electrical effect comprises a positive charge to the first portion of the expandable stent structure. Alternatively, the first electrical effect can comprise a negative charge to the first portion of the expandable stent structure.

In another variation, the method can further include obtaining an impedance measurement using the second portion of the expandable stent structure while the first electrical effect comprises either a negative charge or a positive charge to the first portion of the expandable stent structure.

A variation of the method includes applying energy to the expandable stent structure comprises negatively charging at least a portion of the expandable stent structure.

Variations of the method can include the expandable stent structures that comprise a frame structure forming a plurality of struts, where the frame structure is moveable between a reduce profile and an expanded profile in which a diameter of the frame structure increases; where at least one of the plurality of struts forming the frame structure comprises an electrically conductive material on a support material, the electrically conductive material extending along at least a portion of the strut and being covered with a non-conductive material.

In another variation of the method, the expandable stent structure comprises; at least one electrode formed by an opening in the non-conductive material on the portion of the strut.

Variations of the method can also include using at least a portion of the non-conductive material to create a capacitive effect between at least a portion of the electrically conductive material and the blood vessel.

In variations of the method, the obstruction comprises a blood clot, debris from a blood clot caused during the procedure, plaque, cholesterol, thrombus, a naturally occurring foreign body, a non-naturally occurring foreign body or a combination thereof.

The first electrical effect and/or the second electrical effect can cause altering of movement of a debris from the obstruction. Such altered movement of the debris can comprise electrical attraction between the debris and a portion of the expandable stent structure.

A variation of the method includes applying energy to the expandable stent structure to cause the first electrical effect within the blood vessel comprises cycling energy at various parameters.

In an additional variation of the method, applying energy to the expandable stent structure causes the first electrical effect of increasing coagulation of blood at a portion of the expandable stent structure which causes debris flowing in the blood to adhere to the expandable stent structure.

### BRIEF DESCRIPTION OF THE DRAWINGS

Preferred embodiments of the present invention are hereafter described, by way of non-limiting example only, with reference to the accompanying drawing.

FIG. 1 is a diagrammatic illustration of a system for controlling use of apparatus coupled to an animal or a human.

FIGS. 2A to 2C illustrates an example of a surgical procedure intended to clear the blockage/obstruction from the vessel.

FIG. 3 a diagrammatic illustration showing parts of the system shown in FIG. 1.

FIGS. 4A, 4B, and 5 show examples of medical devices comprising a collapsible and expandable stent 101 with electrodes.

FIG. 6 is a diagrammatic illustration of a medical device located in a vessel.

FIGS. 7A to 7E are diagrammatic variations of medical device of the system.

FIG. 8A is a diagrammatic illustration showing electrode mounting platforms of a medical device of the system shown in FIG. 1.

FIG. 8B is a diagrammatic illustration showing placements of variation of medical devices.

FIGS. 9A-9D illustrate examples of stents or scaffoldings having a plurality of electrodes disposed about the stent body.

FIGS. 10A-10C illustrate an example of integrated or embedded electrodes.

FIGS. 11A-11B show an example of a stent structure fabricated with dimensional variation to impart specific characteristics to the stent.

FIG. 12A illustrates a number of conductive tracks extending in a strut of a stent.

FIGS. 12B to 12E illustrate cross sectional views of some examples of the tracks.

### DETAILED DESCRIPTION OF PREFERRED EMBODIMENTS OF THE INVENTION

The system described herein includes a medical device that is designed for placement within a vessel 202 of an animal or human 110 to engage a clot or obstruction 204 within the vessel 202 to restore blood flow 206 within the vessel 202. The obstruction can comprise a blood clot, debris from a blood clot caused during the procedure, plaque, cholesterol, thrombus, a naturally occurring foreign body, a non-naturally occurring foreign body or a combination thereof. While the present disclosure discusses removal of a clot from a cerebral vasculature, the methods and devices described herein are useful in removal of an obstruction from any vessel within the body, including but not limited to: pulmonary embolism; clots in the legs, arms, etc.; venous and arterial clots.

As shown in FIG. 1, an ischemic stroke occurs when an obstruction 204 blocks blood flow 206 within a region 209 of the cerebral vasculature. The illustration of FIG. 1 shows a clot 204 purely for illustrative purposes to demonstrate the features and characteristics of the system as described below.

FIG. 2A illustrates one example of an initial stage of a surgical procedure intended to clear the blockage/obstruction 204 from the vessel 202. As shown, a physician advances a catheter 222 through the vasculature and into the vessel 202. The tortuosity of the vasculature as well as the reduced size of the vessel 202 can limit the degree to which the physician can advance the catheter 222. The catheter 222 is commonly used to advance a wire 220 through the vessel 202. The wire 220 can then be passed through the obstruction 204. Clearly, any number of procedural variations are within the scope of this disclosure. For example, the wire 220 can be passed between the obstruction 204 and a wall of the vessel 202. Alternatively, the wire 220 can be passed adjacent to the obstruction 204 without passing to a distal region of the obstruction. Alternatively, the wire 220 can be passed to a proximal side of the obstruction (proximal being defined relative to the operator/physician). Alternatively, the use of a wire 220 and/or catheter 222 is not required.

FIG. 2B shows a separate catheter (e.g., a microcatheter) 226 being advanced through the catheter 222 and over the wire. The microcatheter contains a stent-type device (not shown in FIG. 2B) and passes into or through the clot 204. Once positioned, the physician withdraws the microcatheter 224 leaving the stent 100 in place as shown in FIG. 2C. The stent 100 is typically a mesh or strut configuration such that portions of the stent 100 expand into and enmesh within the obstruction 204. The stents can include self-expanding stents or stent structures that require some type of activation to expand into the obstruction 204. It is noted that any adjunct therapeutic substance (e.g., tissue plasminogen activator or “Tpa”) can be delivered to the obstruction 204 or region using any of the catheters.

FIG. 3 illustrates the deployed stent 100 enmeshed with the obstruction 204. As discussed below, the stent 100 can be configured to provide a conducting layer such that power can be applied from a remote source 150 (in this case a disposable unit) through a conducting wire 132 to the stent 100. Alternatively, power can be applied wirelessly where the wire 132 is simply a mechanical tether that allows removal of the stent 100 and obstruction 204.

In any case, the ability to enmesh the stent 100 within the obstruction 204 as well as deliver energy to the stent allows for the power supply 150 to produce an electrical effect within the blood vessel. As noted below, the electrical effect is generally any effect that assists with removal of the obstruction 204 and/or causes debris or other thrombi to become adhered to the stent through electromagnetic attraction to the powered stent 100. The power supply 150 can comprise a disposable power supply or can be a controller that is reusable with various stent/catheter systems. Alternatively, the power supply can comprise a system that inductively or otherwise wirelessly delivers energy to the stent 100.

Ultimately, the stent 100 is manipulated to dislodge at least a portion of the obstruction from the occlusion site and then the stent 100 and captured obstruction are withdrawn from the vessel allowing blood flow to resume within the blood vessel.

In one variation, energy is applied to the entire stent structure 130 to produce a charge, where the charge produces a positive electrical effect within the vessel. For instance, it is believed that positively charging at least a portion of the expandable stent structure/electrodes can prevent accelerate coagulation and clotting of blood and electromagnetic attraction of debris or other thrombi to the stent 100. Such an effect reduces the likelihood that debris caused by dislocation and/or movement of the clot 204 will migrate further downstream in the vasculature. Alternatively, negatively charging at least a portion of the expandable stent structure can prevent clotting and electromagnetic attraction of debris or other thrombi to the stent 100.

As shown, the stent 100 can comprises an expandable stent structure. Such an expandable structure can be self-expanding or activated to expand. The stent 100 can further comprises a frame structure forming a plurality of struts, where the frame structure is moveable between a reduce profile and an expanded profile in which a diameter of the frame structure increases as it engages the obstruction 204.

As discussed below, at least one of the plurality of struts forming the frame structure comprises an electrically conductive material on a support material, the electrically conductive material extending along at least a portion of the strut and being covered with a non-conductive material. FIG. 3 illustrates the stent 100 as having one or more electrodes 131. However, the electrodes can be covered with a material that produces a capacitive effect within the blood vessel. Furthermore, the entire stent 100 can comprise a single electrode (e.g., a single channel). In additional variations, the stent struts can be uniform, meaning there is no increased surface area for an electrode. In such a case, the conductive layer can be covered with a material to allow one or more regions of the device 100 to produce capacitive coupling with blood, the clot, and/or body tissue. Alternatively, or in combination, a power supply can be configured to produce regions of the stent 100 having different charges. For example, as shown in FIG. 3, the stent 100 can be configured to have any number of regions, but is illustrated to have three regions 240, 242, and 244. In such a case, it may be desirable to having distal region 240 (i.e. the portion of the stent 100) distal to the clot 204 to have a specific energy profile, while a portion of the stent that engages the obstruction comprises a separate energy profile 242. Lastly, the portion 244 of the stent 100 that is proximal to the obstruction 204 can have a different energy profile than the first two portions (240, 242). Energy delivery to any portion of the stent 100 can be varied such that the entire stent 100 acts with a single energy profile. For example, it may be desirable to cause the entirety of the stent 100 to have a negative charge which causes debris from the clot 204 to become electrically attracted to the stent.

In another variations, the ability to provide different regions of charge along a stent also allows one or more regions to be used for impedance measurements as an indication of clot adhesion to the stent device. Some regions can be used to attract portions of the clot or debris, while others could probe the environment for impedance changes.

Variations of the stent 100 can include formation of at least one electrode formed by an opening in the non-conductive material on the portion of the strut. In any case, variations of the system are intended to produce an electrical effect that causes an altering of movement of a debris from the obstruction. Such movement can aid in removal of the debris or cause the debris to attach to the stent 100.

The application of energy to the stent 100 can occur prior to deployment of the stent 100 In other variations, applying energy to the expandable stent structure 100 can comprise cycling energy at various parameters before, during, and/or after expansion of the stent 100 into the obstruction.

In yet another variation, applying energy to the expandable stent structure cause the electrical effect of increasing coagulation of blood at a portion of the expandable stent structure which causes debris flowing in the blood to adhere to the expandable stent structure.

1. Medical Device

As shown in FIGS. 4A, 4B, and 5A, the medical device 100 generally includes: a. a collapsible and expandable stent 101; b. a plurality of electrodes 131 coupled to the stent 101; c. electrode lead wires 141 electrically coupled to electrodes 131; d. an olive 112 coupled to the stent 101 by an olive wire 114 for preventing perforation of vessels during implantation; e. implanted chips; f. contacts 151 couple to the lead wires 141 to enable communication between the device 100 to the control unit 12; and g. a stent shaft 121 is used to deploy the device 100.

Electrode lead wires 141 can be electrically connected to at least one electrode and will be wound around the stent strut lattice 108 such that mechanical compression and extension is not interfered with. Electrode wires 141 may be wound around the stent shaft 121, thread through a stylet shaft or may form part of the stent shaft directly. Lead wires 141 will form connections with electrode contacts 151 on the opposite end of the stent shaft to the stent, whereby electrical contact a connector block mechanism 12 enables the connection path with external equipment 16, which included but is not limited to computers, wheelchairs, exoskeletons, robotic prosthesis, cameras, vehicles and other electrical stimulation, diagnostic and measurement hardware and software.

The term electrode 131 is used in this specification to refer to any electrical conductor used to make contact with media in and/or around a blood vessel 103.

A detailed description of the operation of each of these components is set out below.

The Stent

The stent 101 includes a plurality of struts 108 coupled together with strut cross links 109.

In the arrangement shown in FIG. 7a, the device 100 includes nine electrodes coupled to the stent 101 in a linear pattern. As shown, the stent 101 appears flat. The top of the stent 101 may be directly joined to the bottom of the stent 101 or will curve around to meet (without permanent attachment) the bottom of the stent 101.

Alternatively, the device 100 includes a stent with any suitable number of electrodes 131 arranged in any suitable configuration. For example, the electrodes can be configured as follows: the sinusoidal arrangement of electrodes 131 shown in FIG. 7b; the spiral arrangement of electrodes 131 shown in FIG. 7c to enable 360 degree contact of an electrode to the vessel wall once deployed; the reduced amplitude sinusoidal arrangement of electrodes 131 shown in FIG. 7d for increased coverage whilst still ensuring only one stent is at each vertical segment; and the dense arrangement of electrodes shown in FIG. 7e for increased coverage. The stent 101 is laser cut or woven in a manner such that there is additional material or markers where the electrodes 131 are to be placed to assist with attachment of electrodes and uniformity of electrode locations. For example, if a stent 101 was fabricated by laser cutting material away from a cylindrical tube (original form of stent), and, for example, electrodes are to be located at 5 mm intervals on the one axis, then electrode mounting platforms 107, 108 can be created by not cutting these areas from the tube. Similarly, if the stent is made by wire wrapping, then additional material 107, 108 can be welded or attached to the stent wires providing a platform on which to attach the electrodes. Alternatively, stents can be manufactured using thin-film technology, whereby material (Nitinol and or platinum and or other materials or combinations of) is deposited in specific locations to grow or build a stent structure and/or electrode array.

Electrodes

As particularly shown in FIG. 8a, the device 100 includes electrode placements 107 coupled to strut cross links 109. The placements 107 are used to couple the electrodes 131 to the stent. An alternative embodiment of the placements 106 is shown in FIG. 8b. In this embodiment, the placements are circular.

As shown, the electrodes 131 are located on or at the stent cross links 109. Locating the electrodes in these positions allows for changes in shape of the stent 101 (i.e expanding and collapsing) without significantly affecting the integrity of the electrodes. Alternatively, may also be located in between the stent strut crosslinks (not depicted).

To enhance contact and functionality of the device 100, electrodes 131 include the attachment of additional material (shape memory alloy or other conducting material) through soldering, welding, chemical deposition and other attachment methods to the stent 101 including but not limited to: directly on or between the stent struts 108; to lead wires 14 passing from the electrodes 131 to wireless telemetry links or circuitry; and directly to an olive 112 placed on the distal aspect of the device 100 to or stent shafts.

To further enhance the device 100 performance, there may be one or more electrodes 131 per wire strand 141 and there may be one or more strands 141 utilized per device 100. These strands 141 may be grouped to form a bundle 144, which may be woven in alternate sinusoidal paths around the stent struts 108 in the manner shown in FIG. 11. Similarly, there may be one or more wires 141 designated to each electrode 131 and hence there may be one or more electrodes 131 per device 100. Thus, multiple electrodes 131 may be used simultaneously.

Alternatively, the electrodes 131 are made from electrically conductive material and attached to one or more stents, which form the device 100 and allow for multiple positions. In this embodiment, the electrodes 131 are made from common electrically active materials such as platinum, platinum-iridium, nickel-cobalt alloys, or gold, and may be attached by soldering, welding, chemical deposition and other attachment methods to one or more lead wires 141, which may be directly attached to the shape memory shaft(s). The electrodes 131 are preferably one or more exposed sections on the insulated lead wire 141 and the electrode lead wires may be wrapped around one or more shape memory backbones. There may be one or more electrodes and lead wires wrapped around a single shape memory backbone, and, where multiple shape memory backbones are used in the one device, the backbones may have different initial insertion and secondary deposition positions. Thus, they may be used for targeting multiple vessels simultaneously.

Contacts

As particularly shown in FIGS. 4A and 4B, electrode contacts 151 are required to enable connection of the device 100 to external equipment in the situation where wireless circuitry is not employed. The electrode contacts 151 are preferably made from materials similar to those used by the electrodes and will be of similar diameters. The contacts 151 are electrically insulated from each other and will be connected to the electrode lead wires 141 by (but not limited to) conductive epoxy, laser or resistance welding, soldering, crimping and/or wire wrapping.

The contacts 151 are platinum rings or rings of other conductive, biocompatible materials. The contacts can be made from or contain magnetic materials (ie, Neodinium).

The contacts 151 are preferably: (a) between 500 um and 2 mm in diameter; (b) between 500 um and 5 mm in length; and (c) between 10 um and 100 um in thickness.

The contacts 151 are shaped as discs, tubes, parabaloids or other shapes similar to those used for the electrodes 131.

The contacts are placed over non-conducting sleeve (including but not limited to a silicone tube, heat shrink, polymer coating) to assist with electrical insulation of other lead wires and electrode and stent wire, and to assist in retaining shape tubular shape whilst allowing some flexibility.

Preferably, the contacts 151 have a contact to contact separation of between 100 um and 10 mm.

The contacts 151 are formed through wire wrapping of the wires 141.

Preferably, at least one contact 151 is a dummy connector (including but not limited to a metal ring, magnetic ring, plastic tube). A dummy connector in this instance is a connector that is not in electrical contact with an electrode, instead, the purpose is to enable a connection or securing point (ie, through a screw terminal) to the device in a desired location and such that the contacts (connected to electrodes) are not damaged.

The contacts 151 are separated by a non-conductive sleeve (including but not limited to a silicone tube, heat shrink, polymer coating) to reduce electrical noise and prevent contact between superficial lead wires 141.

FIG. 9A also illustrates a variation of a stent 101 that can be fabricated where stent structure comprises an integrated conductive layer that extends through a portion or more of the stent strut 108 and where the electrode 131 is formed through an exposed portion of the integrated conductive layer. Such a stent configuration, as described in detail below, permits a stent 101 electrode 131 assembly, which embeds electrodes and conductive electrode tracks into the stent lattice or strut itself. Such a construction reduces or eliminates the requirement to use fixation methods (i.e., adhesives, glues, fasteners, welds, etc.) to mount electrodes to the body of the stent. Such a construction further reduces or eliminates the need to further weld or electrically connect electrodes to wires. Another benefit is that conventional wire-connected-electrodes require accommodation of the wires about the stent struts and through the body of the stent.

FIG. 9B illustrates a stent structure 101 with integrated electrodes 131, where the expandable stent structure is coupled to a shaft 121 at a distal end 146. The shaft, as described herein, can electrically couple the electrodes 131 to one or more control units (not shown) as described herein. In one example, the shaft 121 can comprise a guidewire, push wire other tubular structure that contains wires or conductive members extending therein and are coupled to the conductive layer of the stent at the distal end 146. Alternatively, FIGS. 9C and 9D shows a variation of stents 101 that can be fabricated such that the shaft 121 is part of or integral with the expandable stent structure, where the conductive layer extends through a portion or all of the stent to the shaft 121. Such a construction further eliminates the need for joining the shaft to the expandable stent structure at the working end of the stent. Instead, the joining of the expandable stent structure (forming the shaft) to a discrete shaft can be moved proximally along the device. Such a construction allows the working end of the stent and shaft to remain flexible. The expandable stent structures shown in FIGS. 9C and 9D can also include an optional reinforced section 62 as discussed above. FIG. 9C further illustrates a hollow shaft 121, which allows insertion of a stylet 123 therethrough to assist in positioning of the device or permits coupling of wires or other conductive members therethrough. Furthermore, the shaft 121 can include any number of features 119 that improve flexibility or pushability of the shaft through the vasculature.

The electrical connection of the electrodes 131 to leads extending through the device can be accomplished by the construction of one or more connection pads (similar in construction to the electrodes described below) where the size of the pads ensures sufficient contact with the wire/lead, the type of pads ensures robustness and reduces track fatigue when crimped and attached. The section containing the pads can be compressed into a tube at, for example, distal section 146 to enable insertion of a cable 121.

In certain variations, the connection pads should be able to feed through the catheter. Furthermore, the connection pads 132 can include one or more holes or openings that enable visual confirmation that the pads are aligned with contacts on the lead. These holes/openings also enables direct/laser welding or adhesion of the contact leads (inside tube 121) and the contact pads (on the inside of the tube spanning through the hole to the outside)

In one example, a coaxial-octofilar cable (i.e. an inner cable with 8 wires positioned inside an outer cable having 8 wires) is used to enhance fatigue resistance and to ensure that wires can fit within constraints (i.e., can be inserted through a sufficiently small catheter, and can have an internal stylet as required).

FIGS. 9A-9D illustrate one example of a stent structure 101 constructed with an embedded electrode and conductive path. FIG. 9A illustrates an example of a stent structure 101 in a planar configuration with electrodes 138 in a linear arrangement for purposes of illustration only. Clearly, any configuration of electrodes is within the scope of this disclosure. Specifically, in those variations of stent structures useful for neurological applications, the expandable stent structure can comprise a diameter that is traditionally greater than existing neurological stents. Such increased diameter can be useful due to the expandable stent structure being permanently implanted and while requiring apposition of electrodes against the vessel/tissue wall. Moreover, in some variations, the length of such stent structures can include lengths up to and greater than 20 mm to accommodate desired placement along the human motor cortex. For example, variations of the device require a stent structure that is sufficiently long enough to cover the motor cortex and peripheral cortical areas. Such lengths are not typically required for existing interventional devices aimed at restoring flow or addressing aneurysms or other medical conditions. In addition, in certain variations, the electrical path between certain electrodes can be isolated. In such a case, the electrically conductive material 50 can be omitted from certain stent struts to form a pattern that allows an electrode to have an electrical conduction path to a contact pad or other conductive element but the electrical conduction path is electrically isolated from a second electrode having its own second electrically conductive path.

Placement of the electrodes in a specific pattern (e.g., a corkscrew configuration or a configuration of three linear (or corkscrew oriented) lines that are oriented 120 degrees from each other) can ensure a deployed electrode orientation that directs electrodes towards the brain. Once implanted, orientation is not possible surgically (i.e., the device will be implanted and will be difficult if not impossible to rotate). Therefore, variations of the device will be desirable to have an electrode pattern that will face towards the desired regions of the brain upon delivery.

Electrode sizing should be of a sufficient size to ensure high quality recordings and give large enough charge injection limits (the amount of current that can be passed through the electrodes during stimulation without damaging the electrodes which in turn may damage tissue). The size should also be sufficient to allow delivery via a catheter system.

FIGS. 10B and 10C illustrates a cross-sectional view of the expandable stent structure of FIG. 10A taken along line 10B-10B to further illustrate one variation of a manufacturing technique of using MEMS (microelectrical mechanical systems) technology to deposit and structure thin film devices to fabricate a stent structure with electrodes and a conductive path embedded into the stent lattice or struts. The spacing of the struts in FIGS. 10B and 10C are compressed for illustrative purposes only.

As discussed above, embedding the electrode and conductive path presents advantages in the mechanical performance of the device. Furthermore, embedding of electrodes provides the ability to increase the number of electrodes mounted on the structure give that the conductive paths (30-50 μm×200-500 nm) can be smaller than traditional electrode wires (50-100 μm).

Manufacture of thin-film stents can be performed by depositing Nitinol or other superelastic and shape memory materials (or other materials for deposition of electrodes and contacts (including but not limited to gold, platinum, iridium oxide) through magnetron sputtering in a specific pattern (56) using a sacrificial layer (58) as a preliminary support structure. Removal of the support structure (54) enables the thin film to be further structured using UV-lithography and structures can be designed with thicknesses corresponding with radial force required to secure the electrodes against a vessel wall.

Electrical insulation of electrodes is achieved by RF sputtering and deposition of a non-conductive layer (52) (eg, SiO) onto the thin-film structure (54). Electrodes and electrode tracks (50) are sputter deposited onto the non-conductive layer (using conductive and biomedically acceptable materials including gold, Pt, Ti, NiTi, PtIr), with an additional non-conductive layer deposited over the conductive track for further electrical isolation and insulation. As shown, conducting path 50 is left exposed to form the electrode 138 (similarly, a contact pad area can remain exposed). Finally, the sacrificial layer 56 and substrate are removed leaving the expandable stent structure 101 as shown in FIG. 9C.

In certain variations where the base structure 54 comprises superelastic and shape-memory materials (i.e. Nitinol), the expandable stent structure 101 can be annealed in a high vacuum chamber to avoid oxidation during the annealing process. During heat treatment, the amorphous Nitinol structure 54 crystallizes to obtain superelasticity and can be simultaneously shape set into a cylindrical or other shape as desired. The structure 101 can then be heat treated.

FIG. 11A, which is a partial sectional view of taken along lines 11A-11A of FIG. 11B, illustrate an additional variation of a stent structure 101 fabricated via MEMS technology where one or more stent struts 108 can be dimensionally altered to impart desired structural or other aspects to the expandable stent structure 101. For example, in the illustrated variation, certain stent struts 108 are dimensionally altered such that the support material 60 comprises a greater thickness than adjacent stent structures 108. However, such dimensional variation is not limited to thickness but can also include width, shape, etc.

FIG. 11B illustrates the expandable stent structure 101 resulting from the dimensionally altered struts resulting in a sinusoidal section 62 of the expandable stent structure 101 that comprises a greater stiffness (resulting from the increased thickness). Such a configuration allowing the stent device to be pushed through a catheter rather than conventional requirements to be unsheathed (where the sheath is pulled back over the stent). Conventional stents are made from a thin lattice of Nitinol diamonds or cells. This sinusoidal section 62 can function like a backbone and gives forward pushing strength to the device without restricting super-elasticity and the ability for the stent to compress and expand. Clearly, any number of variations of dimensionally altered strut sections are within the scope of this disclosure.

FIG. 12A illustrates an example of s stent structure 101 having struts 108 designed as discussed above. FIG. 12A also shows a magnification of the struts 108 where one or more electrically conductive channels 252, 254 can extend along a length of the strut. Although two channels 252, 254 are shown separated by an electrically insulative divider 256, a single channel can extend in a strut 108 or more than 2 channels.

In the variation shown in FIG. 12A, the channels 252, 254 can function as electrode tracks such that any length of the stent (e.g., various portions as noted above, or the entire length of the stent 101) can be used for attraction along the channels 252, 254, while the remainder of the stent 101 has on electrical charge. Such a construction increases the useful/usable length of the expandable stent structure 101. Again, the variation shown in FIG. 12A is one variation that allows a single track or parallel tracks (where parallel tracks 252, 254 can opposite poles) through which to attract debris.

The tracks can remain uninsulated and be used to attract debris across the length of the stent and over the entire circumference of the stent. In another variation, a single track could be used, or multiple tracks can be used (to increase the relative negativity or area).

FIGS. 12B to 12C illustrate cross sectional illustrations of a strut formed from a base or support material 54, which often forms the non-conductive portion of the strut and a conductive material 50 as discussed above. The debris 208 is believed to be attracted to the conductive material 50 or charge created by the conductive material 50 as noted above. FIG. 12B illustrates a conductive portion 50 protruding beyond the support 54. FIG. 12C shows the conductive portion 50 at or below a surface of the support 54. FIG. 12D shows the conductive material 50 recessed within the support 54 (where the conductive material 50 can optionally extend through the support 54). FIG. 12E illustrates a plurality of fins 50 or tracks of conductive material 50 extending along the support 54.

Again, the tracks disclosed above can be made in any number of configurations, including having them recessed into the scaffold or as fins to increase the volume of debris that can be attracted without increasing the volume and difficulty of debris removal. (i.e., when the stent/catheter is removed, a large amount of debris will impact the stent compression and may make removal difficult. Having set or predetermined areas where the debris is attracted will improve efficiency of getting the debris without impacting the ability to remove the device). Again, the images shown in FIG. 12B to 12E are cross sections and the tracks/conductive material can extend a significant portion of the stent.

Many modifications will be apparent to those skilled in the art without departing from the scope of the present invention.

The reference to any prior art in this specification is not, and should not be taken as, an acknowledgment or any form of suggestion that the prior art forms part of the common general knowledge in Australia

In this specification and the claims that follow, unless stated otherwise, the word “comprise” and its variations, such as “comprises” and “comprising”, imply the inclusion of a stated integer, step, or group of integers or steps, but not the exclusion of any other integer or step or group of integers or steps.

References in this specification to any prior publication, information derived from any said prior publication, or any known matter are not and should not be taken as an acknowledgement, admission or suggestion that said prior publication, or any information derived from this prior publication or known matter forms part of the common general knowledge in the field of endeavour to which the specification relates.

We claim:

1. A method for restoring blood flow in a blood vessel occluded by an obstruction at an occlusion site in the blood vessel, the method comprising:
advancing a microcatheter into the blood vessel, the microcatheter containing an expandable stent structure;
deploying the expandable stent structure within the blood vessel such that a portion of the expandable stent structure embeds into the obstruction;
applying energy to the expandable stent structure to cause a first electrical effect along a first portion of the expandable stent structure and a second electrical effect along a second portion of the expandable stent structure, within the blood vessel, where the first electrical effect varies from the second electrical effect and where at least the first electrical effect or the second electrical alters an attraction of the obstruction to the expandable stent structure;
manipulating the expandable stent structure to dislodge at least a portion of the obstruction from the occlusion site; and
withdrawing the expandable stent structure and at least the portion of the obstruction from the vessel allowing blood flow to resume within the blood vessel.

2. The method of claim 1, where the first electrical effect comprises a positive charge to the first portion of the expandable stent structure.

3. The method of claim 1, where the first electrical effect comprises a negative charge to the first portion of the expandable stent structure.

4. The method of claim 1, further comprising obtaining an impedance measurement using the second portion of the expandable stent structure while the first electrical effect comprises either a negative charge or a positive charge to the first portion of the expandable stent structure.

5. The method of claim 1, where applying energy to the expandable stent structure comprises negatively charging at least a portion of the expandable stent structure.

6. The method of claim 1, wherein the expandable stent structure comprises a frame structure forming a plurality of struts, where the frame structure is moveable between a reduce profile and an expanded profile in which a diameter of the frame structure increases;
where at least one of the plurality of struts forming the frame structure comprises an electrically conductive material on a support material, the electrically conductive material extending along at least a portion of a strut of the plurality of struts and being covered with a non-conductive material.

7. The method of claim 6, wherein the expandable stent structure comprises;
at least one electrode formed by an opening in the non-conductive material on the portion of a strut of the plurality of struts.

8. The method of claim 6, where the first electrical effect comprises at least a portion of the non-conductive material creating a capacitive effect between at least a portion of the electrically conductive material and the blood vessel.

9. The method of claim 1, where the obstruction comprises a blood clot, debris from a blood clot caused during a procedure, plaque, cholesterol, thrombus, a naturally occurring foreign body, a non-naturally occurring foreign body or a combination thereof.

10. The method of claim 1, wherein the first electrical effect causes altering of movement of a debris from the obstruction.

11. The method of claim 9, where altering of movement of the debris comprises electrical attraction between the debris and a portion of the expandable stent structure.

12. The method of claim 1, wherein applying energy to the expandable stent structure to cause the first electrical effect within the blood vessel comprises cycling energy at various parameters.

13. The method of claim 1, wherein applying energy to the expandable stent structure cause the first electrical effect of increasing coagulation of blood at a portion of the expandable stent structure which causes debris flowing in the blood to adhere to the expandable stent structure.
